(19) United States
(12) Patent Application Publication
(10) Pub. No.: US 2012/0004555 A1
(43) Pub. Date: Jan. 5, 2012

(54) METHOD OF STRETCHING ORGANIC PIEZOELECTRIC MATERIAL, METHOD OF MANUFACTURING ORGANIC PIEZOELECTRIC MATERIAL, ULTRASONIC TRANSDUCER, ULTRASONIC WAVE PROBE AND ULTRASONIC WAVE MEDICAL IMAGE DIAGNOSIS DEVICE

(75) Inventors: Kenji Ohnuma, Tokyo (JP); Hiromi Akahori, Tokyo (JP); Yuichi Nishikubo, Kanagawa (JP)

(73) Assignee: Konica Minolta Medical & Graphic, Inc., Tokyo (JP)

(21) Appl. No.: 13/256,781

(22) PCT Filed: Mar. 5, 2010
(86) PCT No.: PCT/JP2010/053633

§ 371 Date: Sep. 15, 2011

(30) Foreign Application Priority Data
Mar. 18, 2009 (JP) 2009-066225

(51) Int. Cl. A61B 8/00; G10K 9/122; H01L 41/22

(52) U.S. Cl. 600/459; 29/25.35; 310/334

(57) ABSTRACT

Provided is a stretching method of an organic piezoelectric material which sequentially performs a primary stretching step for carrying out primary stretching of an organic piezoelectric material which has not been stretched, a heat treatment step for heat treating the organic piezoelectric material subjected to primary stretching, and a cooling step for carrying out secondary stretching of the heat treated organic piezoelectric material while the organic piezoelectric material is cooled down to the room temperature, and is characterized in that tension is applied continuously to the organic piezoelectric material from the primary stretching step to the cooling step without releasing the tension, and heat treatment is carried out while keeping the tension in a range of 0.1-500 kPa. A stretching method and a production method for producing an organic piezoelectric material exhibiting excellent planarity, machining characteristics and piezoelectric characteristics and suitable for high frequency and broadband are thereby provided, and an ultrasound transducer using the organic piezoelectric material produced by the method, and an ultrasound medical image diagnosis device are also provided.

### FIELD OF THE INVENTION

The present invention relates to a method for stretching an organic piezoelectric material and a method for manufacturing an organic piezoelectric material employing in an ultrasonic transducer which is suitable for high frequency and broad band, an ultrasonic transducer, an ultrasonic wave probe and an ultrasonic wave medical diagnostic imaging system employing the organic piezoelectric material manufactured by using thereof.

### BACKGROUND OF THE INVENTION

Generally, a sonic wave of 16 kHz or more is collectively called an ultrasonic wave. The ultrasonic wave makes it possible to check the inside of an object nondestructively and harmlessly and is utilized in various fields such as detection of defects, diagnosis of disease, and others. One of the applications is an ultrasonic wave diagnostic system, in which the inside of an examinee is scanned by an ultrasonic wave to form an image of the inside of the examinee based on a reception signal generated from a reflection ultrasonic wave (echo) from the inside. This ultrasonic wave diagnostic system employs an ultrasonic wave probe which transmits an ultrasonic wave to an examinee and receives an ultrasonic wave from the examinee. This ultrasonic wave probe employs an ultrasonic wave transmission and reception element which is provided with an oscillator which vibrates mechanically based on a transmission signal and generates an ultrasonic wave and produces a reception signal by receiving a reflection ultrasonic wave generated from difference of the acoustic impedance in the inside of the examinee.

In recent years, a harmonic imaging technique has been studied and developed which forms an in-examinee image employing the harmonic frequency component of an ultrasonic wave transmitted from an ultrasonic wave probe to the examinee instead of the frequency (fundamental frequency) component of the transmitted ultrasonic wave. This harmonic imaging technique has many advantages in that (1) the side lobe level is small as compared with that of the fundamental frequency component and the S/N ratio (signal to noise ratio) is improved, resulting in improved contrast resolution, (2) the high frequency reduces the beam width, resulting in improved resolution in the lateral direction, (3) the low sound pressure and small sound pressure fluctuation at a short distance minimizes the multiple reflection, and (4) the attenuation beyond focus, which is at the same level as that of the fundamental wave provides high depth-speed as compared with the case where a high frequency wave is used as the fundamental wave.

The ultrasonic wave probe for the harmonic imaging requires a broad frequency band ranging from the frequency of a fundamental wave to the frequency of a harmonic wave. The frequency region on the lower frequency side is employed for transmission to transmit the fundamental wave, while the frequency region on the higher frequency side is employed for reception to receive the fundamental wave (see for example, Patent Document 1).

The ultrasonic wave probe disclosed in this Patent Document 1 is one which, when applied to an examinee, transmits an ultrasonic wave to the inside of the examinee, and receives an ultrasonic wave returned by reflection from them. The ultrasonic wave probe is provided with a first piezoelectric layer composed of a plurality of arranged first piezoelectric elements having a predetermined first acoustic impedance, and the first piezoelectric layer has a role in transmitting a fundamental wave comprised of an ultrasonic wave with a predetermined center frequency to an examinee and receiving a fundamental wave among the ultrasonic waves returned by reflection from the inside of the examinee. This ultrasonic wave probe is also provided with a second piezoelectric layer composed of a plurality of arranged second piezoelectric elements having a predetermined second acoustic impedance smaller than the predetermined first acoustic impedance, and the second piezoelectric layer has a role in receiving a harmonic wave among the ultrasonic waves returned by reflection from the inside of the examinee. Herein, the second piezoelectric layer is overlapped on the entire surface of the first piezoelectric layer on the side on which the ultrasonic wave probe is applied to the examinee. The ultrasonic wave probe having the structure as described above can transmit and receive an ultrasonic wave in a broad frequency band.

The fundamental wave in harmonic imaging is preferably a sonic wave having the possible narrowest band width. As a piezoelectric element, so-called ceramics inorganic piezoelectric materials are widely used which include a single crystal of quartz, LiNbO₃, LiTaO₃ or KNbO₃; a thin film of ZnO or AlN; and calcination products of Pb(Zr, Ti)O₃ and the like, each subjected to polarization treatment. However, these inorganic materials are not suitable for application to a piezoelectric element detecting a reception wave on a higher frequency side requiring sensitivity to a broader band region. As a piezoelectric element suitable for high frequency and broad band, an organic piezoelectric material employing an organic polymer material such as polyvinylidene fluoride (hereinafter also referred to as “PVDF”) is known (see for example, Patent Document 2). This organic piezoelectric material is flexible, and easy to form a thin film, a large area or a long length, as compared with inorganic piezoelectric materials, and therefore, has advantages of manufacturing those in any shape or structure.

However, the element composed of the organic piezoelectric material has insufficient piezoelectric characteristic as compared with one composed of the inorganic piezoelectric material. In order to enhance a molecular orientation and an amount of polarization, it is known to be effective to subject additional treatment such as a stretching of the film, heat-treatment below melting point, or combination thereof (see for example, Patent Documents 2 and 3). However, when an organic piezoelectric material having PVDF as the main component is manufactured by using these known methods, while piezoelectric property is improved, due to its high cristallinity (see for example, Patent Document 4), an organic piezoelectric material not only looses its flexibility which is the advantage thereof and but becomes brittle. Further, since PVDF has a glass transition temperature below a room temperature, molecular motion cannot be thoroughly frozen when it is cooled to the room temperature from heat-treatment temperature, resulting in causing the film deformation over time, while reducing a residual stress inside. Namely, it was found new problems as specific to an ultrasonic wave probe for an ultrasonic wave diagnostic system such as lowering receiving sensitivity or lowering electrical breakdown strength, as well as being insufficient processing suitability (see for example, Patent Documents 5 and 6).

### PRIOR TECHNICAL DOCUMENT

### Patent Document

Patent Document 1: Japanese Registration Patent No. 4125416
Patent Document 2: Unexamined Japanese Patent Application (hereinafter, refers to JP-A) No. 60-217674
Patent Document 3: JP-A No. 2003-80593
Patent Document 4: JP-A No. 8-36917
Patent Document 5: JP-A No. 5-42592
Patent Document 6: JP-A No. 2008-174577

### SUMMARY

### Problems to be Solved by the Present Invention

The present invention has been made in view of the above. An object of the present invention is to provide a method for stretching an organic piezoelectric material and a method for manufacturing an organic piezoelectric material employing in an ultrasonic transducer which exhibits excellent flatness, processing suitability and piezoelectric characteristic, and is suitable for high frequency and broad band, the ultrasonic transducer employing the organic piezoelectric material, and an ultrasonic wave medical diagnostic imaging system.

### Means to Solve the Problems

The above object of the present invention can be attained by any one of the following constitutions.

1. A method for stretch-treating an organic piezoelectric material comprising sequential steps of primary stretching an organic piezoelectric material which has not been stretched, heat treating the organic piezoelectric material subjected to primary stretching, and secondary stretching the heat treated organic piezoelectric material while cooling down the organic piezoelectric material to the room temperature, wherein tension is applied continuously to the organic piezoelectric material from the primary stretching step to the cooling step without releasing the tension, and heat treatment is carried out while keeping the tension in a range of 0.1-500 kPa.
2. The method for stretching an organic piezoelectric material of item 1, wherein heat treating temperature is in the range of from 100 to 140° C., and heat treating time is in the range of from 30 minutes to 10 hours.
3. The method for stretching an organic piezoelectric material of item 2, wherein primary stretching is carried out mono-axially or bi-axially by a stretching factor from 2 to 10, and secondary stretching is carried out by not more than 10% in the longitudinal direction.
4. The method for stretching an organic piezoelectric material of any one of items 1 to 3, wherein the organic piezoelectric material comprises a copolymer of vinylidene fluoride and triflioroethylene, and a content ratio of vinylidene fluoride and triflioroethylene is in the range of from 95 to 60 mol % and in the range of from 5 to 40 mol %, respectively.
5. A method for manufacturing the organic piezoelectric material comprising a step of polarizing the organic piezoelectric material manufactured by the method of any one of items 1 to 4.
6. An ultrasonic transducer comprising the organic piezoelectric material manufactured by the method of item 5 and an electrode.
7. An ultrasonic wave probe comprising the ultrasonic transducer of item 6.
8. An ultrasonic wave medical diagnostic imaging system comprising an electric signal generating device, an ultrasonic wave probe in which a plurality of oscillators are arranged which receive the electric signal and transmit an ultrasonic wave to an examinee and generate a reception signal according to a reflection wave returned from the examinee and an image processing device which forms an image of the examinee according to the reception signal generated by the ultrasonic wave probe, wherein the ultrasonic wave probe comprises an oscillator for ultrasonic wave reception comprising a piezoelectric material for ultrasonic wave reception, and one or both of the ultrasonic transducers is the ultrasonic transducer of item 6.

### Effects of the Invention

The above means of the present invention can provide a method for stretching an organic piezoelectric material and a method for manufacturing an organic piezoelectric material employing in an ultrasonic transducer which exhibits excellent flatness, processing suitability and piezoelectric characteristic, and is suitable for high frequency and broad band, the ultrasonic transducer employing the organic piezoelectric material, and an ultrasonic wave medical diagnostic imaging system.

### BRIEF DESCRIPTION OF THE DRAWING

FIG. 1 is a schematic drawing showing the structure of the main section of an ultrasonic wave probe.

FIG. 2 is a schematic drawing showing the example of the change of tension during each step.

### PREFERRED EMBODIMENT OF THE INVENTION

The present invention is a method for stretch-treating an organic piezoelectric material comprising sequential steps of primary stretching an organic piezoelectric material which has not been stretched, heat treating the organic piezoelectric material subjected to primary stretching, and secondary stretching the heat treated organic piezoelectric material while cooling down the organic piezoelectric material to the room temperature, wherein tension is characterized by being applied continuously to the organic piezoelectric material from the primary stretching step to the cooling step without releasing the tension, and heat treatment is carried out while keeping the tension in a range of 0.1-500 kPa. This characteristic is a technical characteristic common through claims 1 through 8.

As the embodiment for the present invention, in view of effect of the present invention, while the tension is kept in 0.1-500 kPa, heat treating temperature is preferable in the range of from 100 to 140° C., and heat treating time is preferable in the range of from 30 minutes to 10 hours, and secondary stretching is carried out during cooling down to the room temperature by not more than 10% in the longitudinal direction.

Further preferable embodiment is that the organic piezoelectric material comprises a copolymer of vinylidene fluoride and triflioroethylene, and a content ratio of vinylidene fluoride and triflioroethylene is in the range of from 95 to 60 mol % and in the range of from 5 to 40 mol %. Further, electromechanical coupling coefficient of the organic piezoelectric material is preferable 0.3 or more.

The method for stretch-treating an organic piezoelectric material of the present invention is the method in which the organic piezoelectric material which has not been stretched is subjected to heat treatment after the primary stretching step, wherein the tension is not released (without being 0) and heated while keeping the tension in the range of 0.1 to 500 kPa along with the longitudinal stretching direction from finishing the primary stretching step to finishing the heat treatment, and then further stretching is carried out during cooling down to the room temperature.

Herein, while the tension is kept in 0.1-500 kPa, heat treating temperature is preferable in the range of from 100 to 140° C., and heat treating time is preferable in the range of from 30 minutes to 10 hours. Subsequently, further stretching is carried out during the organic piezoelectric material being cooled down to the room temperature by not more than 10% in the longitudinal stretching direction.

When the organic piezoelectric material related to the present invention is employed to the ultrasonic transducer, it is preferable to manufacture the organic piezoelectric material so as to have the long side of the ultrasonic transducer in perpendicular to the stretch treatment direction. Further, it is also preferable that the organic piezoelectric material is manufactured to have the long side of the ultrasonic transducer parallel to the stretch treatment direction.

The ultrasonic transducer can be employed preferably to an ultrasonic wave medical diagnostic imaging system. In this case, it is preferable that the ultrasonic wave medical diagnostic imaging system comprises a member which generates an electrical signal, an ultrasonic wave probe in which a plurality of oscillators are arranged which transmits an ultrasonic wave to an examinee by receiving the electrical signal and receives an ultrasonic wave reflected from the examinee as an echo signal, and an image processing member which generates an image of the examinee according to the received signal generated by the ultrasonic wave probe, wherein the ultrasonic wave probe comprises both of ultrasonic transducers for a transmission and for a reception, and one or both of the ultrasonic transducers is the ultrasonic transducer of the present invention.

Next, the invention, its constituent and the preferred embodiment of the present invention will be explained in detail below.

### (Ultrasonic Transducer)

The ultrasonic transducer of the present invention is characterized in that it is an ultrasonic transducer used in an ultrasonic wave probe (probe) for an ultrasonic wave medical diagnostic imaging system equipped with an ultrasonic transducer for ultrasonic wave transmission and for ultrasonic wave transmission.

The ultrasonic transducer of the present invention has a structure in which a layer (or film) formed from a piezoelectric material in the form of a film (hereinafter also referred to as a piezoelectric material layer or a piezoelectric material film) is inserted between a pair of electrodes. A plurality of ultrasonic transducers is arranged, for example, in one dimensionally, thereby obtaining an ultrasonic wave probe.

The probe has a function of driving a specific number of oscillators in the longitudinal direction in the plurality of ultrasonic transducers arranged to irradiate the site to be examined in an examinee with convergent ultrasonic wave beams, receive the ultrasonic wave reflection echo returned from the site, and convert the reflection echo to an electric signal.

### (Organic Piezoelectric Material)

As organic piezoelectric material of the piezoelectric material constituting the ultrasonic transducer of the present invention, both low molecular material and polymeric material may be employable. Low molecular organic piezoelectric material include phthalate ester based compounds, sulfenamide based compounds and organic compounds having phenol skeleton. Polymeric organic piezoelectric material include polyvinylidene fluoride or polyvinylidene fluoride based copolymer, polyvinylidene cyanide or polyvinylidene cyanide based copolymer, nylon having uneven number such as nylon 9 or nylon 11, aromatic nylon, alicyclic nylone, polyhydroxy carbonic acid such as polylactic acid, polyhydroxy butyrate, cellulose based derivatives, or poly urea. In view of good piezoelectric characteristic, processing suitability or availability, polymeric organic piezoelectric material having vinylidene fluoride as a main component is required.

Specifically, the polymeric material is required to be a homopolymer of vinylidene fluoride or a copolymer containing vinylidene fluoride as a main component, each containing a CF₂ group exhibiting a large dipole moment. Examples of a second component in the copolymer other than vinylidene fluoride include tetrafluoroethylene, trifluoroethylene, hexafluoropropane and chlorofluoroethylene.

For example, in a vinylidene fluoride/trifluoroethylene copolymer, the electromechanical coupling coefficient (piezoelectric effect) in the thickness direction varies due to the copolymerization ratio and therefore, the copolymerization ratio of the vinylidene fluoride in the copolymer is preferably from 60 to 99 mol %, and more preferably from 85 to 99 mol %.

A copolymer of 85-99 mol % of vinylidene fluoride and 1-15 mol % of perfluoroalkyl vinyl ether, perfluoroalkoxyethylene or perfluorohexaethylene restrains a transmission basic wave and results in enhanced sensitivity to a harmonic component reception in combination of an inorganic piezoelectric element for transmission and an organic piezoelectric element for reception.

The polymer piezoelectric material described above has advantage to be provided an oscillator applied to transmission and reception of higher frequency due to be formed into a thin film as compared with an inorganic piezoelectric material formed from ceramics.

In the present invention, the organic piezoelectric material is characterized in that it has a relative dielectric constant in the thickness resonance frequency of from 10 to 50. Adjustment of the relative dielectric constant can be carried out by adjustment of the number of a polar functional group such as a CF₂ group or a CN group contained in a compound constituting the organic piezoelectric material, the composition or the polymerization degree or by polarization treatment described later.

The organic piezoelectric material film constituting the ultrasonic transducer of the present invention can be a laminate in which a plurality of polymeric materials is multi-layered. In addition to the polymeric materials described above, polymeric materials having a relatively low relative dielectric constant as shown below can be used in combination as the polymeric materials to be multi-layered in the laminate.

In the following examples, the figures in the parentheses represent a relative dielectric constant of the polymeric materials (resins). Examples of the polymeric materials include methyl methacrylate resin (3.0), acrylonitrile resin (4.0), acetate resin (3.4), aniline resin (3.5), aniline formaldehyde resin (4.0), aminoalkyl resin (4.0), alkyd resin (5.0), nylon 6-6 (3.4), ethylene resin (2.2), epoxy resin (2.5), vinyl chloride resin (3.3), vinylidene chloride resin (3.0), urea formaldehyde resin (7.0), polyacetal resin (3.6), polyurethane (5.0), polyester resin (2.8), polyethylene (low pressure) (2.3), polyethylene terephthalate (2.9), polycarbonate resin (2.9), melamine resin (5.1), melamine formaldehyde resin (8.0), cellulose acetate (3.2), vinyl acetate resin (2.7), styrene resin (2.3), styrene butadiene rubber (3.9) and ethylene fluoride resin (2.0).

It is preferred that the polymeric materials having a low relative dielectric constant as shown above are properly selected according to various objects, for example, for the purpose of adjusting the piezoelectric characteristic or increasing physical strength of the organic piezoelectric material layer.

### (Manufacturing Method of Organic Piezoelectric Material)

The organic piezoelectric material of the present invention comprises the polymeric material as the main component and forms film which can be stretched under a temperature from the room temperature to 10° C. lower than melting point. The organic piezoelectric material is manufactured by stretching bi-axially or mono-axially (primary stretching) first, then heating while keeping the tension in the uniform range described above, subsequently by carrying out further stretching (secondary stretching) during cooling down to the room temperature.

When the organic piezoelectric material having vinylidene fluoride is used as an oscillator, the material is formed into a film, and provided with a surface electrode for inputting an electric signal.

The film formation can be carried out according to a general method such as a melting method or a casting method. It is known that a vinylidene fluoride-trifluoroethylene copolymer film itself can form a crystal having polarity by only making a film.

### (Primary Stretching Step)

As the stretching methods, various known methods can be employable. For example, a solution in which the polymeric materials described above are dissolved in an organic solvent such as methyl ethyl ketone (MEK) is cast on a substrate such as a glass plate, dried by evaporating the solvent at ordinary temperature to obtain a film with a predetermined thickness, and stretched by a predetermined factor at room temperature. The stretching can be carried out mono-axially or bi-axially, provided that the organic piezoelectric material film in a predetermined form is not destroyed. The stretching factor is preferably from 2 to 10, and preferably from 2 to 6.

In a vinylidene fluoride-trifiuoroethylene copolymer and/or a vinylidene fluoride-tetrafluoroethylene copolymer, the use of a polymeric piezoelectric material exhibiting a melt flow rate of not more than 0.03 g/min at 230° C., preferably not more than 0.02 g/min and more preferably not more than 0.01 g/min can provide a highly sensitive piezoelectric material thin layer.

### (Heat Treatment Step)

In heat treatment step, the primary stretched organic piezoelectric material is subjected to heat treatment without releasing the tension (without being 0) after the primary stretching step and with keeping the tension in the range of 0.1 to 500 kPa along with the longitudinal stretching direction.

In the present invention, after primary stretching, the tension is kept without releasing preferably in 0.1 kPa or more.

As the heat treatment method of the organic piezoelectric material in the present invention, a method is preferred in which a film of the organic piezoelectric material is allowed to stand around at a temperature, the upper limit of which is 10° C. lower than the melting point of the film while holding the both ends of the film by a chuck or a clip, in order to apply heat effectively or uniformly to the inside of the film.

A method which heats the film by bringing the film in direct contact with a heat source such as a heated plate is undesired, since a material which causes contraction on heat application impairs planarity of the film.

The organic piezoelectric material containing polyvinylidene fluoride as a main component has a melting point of from 150° C. to 180° C., and therefore, it is preferred that it is heat treated at not less than 100° C. and not more than 140° C.

The effect is seen when the heat treatment is carried out for 30 minutes or more, and the longer the heat treatment time is, the more the crystal growth promotes. Since the crystal growth saturates with time, the heat treatment time is actually to around 10 hours, and at most 24 hours.

Herein, the tension along with the longitudinal stretching direction refers to the tension which has direction parallel to the direction of the tension applied in the stretching step.

In view of planarity, the tension during heat treatment is required in the range of from 0.1 to 500 kPa.

### (Cooling Step)

Cooling step is a step in which the heat treated organic piezoelectric material is cooling down to the room temperature and secondary stretching treatment is carried out during cooling down to the room temperature.

The secondary stretching treatment is a relaxation treatment.

The relaxation treatment herein referred to means one which varies stress applied to the both ends of the film while following contraction or expansion force to which the film is subjected in the process in which the heat treatment is carried out, followed by cooling to the room temperature. The relaxation treatment, as long as the planarity of the film is not impaired by film relaxation or the breakage of the film does not occur due to too much stress, may be conducted to contract the film by relaxation of stress or to broaden the film in the direction applying tensile force so as not to cause stretching.

According to the present invention, the second step stretching is carried out by 10% in length, and in the case when the film extends during cooling, around at most 10% while following relaxation, provided that the direction of stretching is defined as plus. In the present invention, a treatment in which stretching chuck is moved to the extent of stretching film taut or taking up slack is referred to as the secondary stretching treatment (the second step stretching).

The example of the change of tension during each step is shown in FIG. 2. In FIG. 2, vertical axis represents tension, horizontal axis represents time, a represents primary stretching step, b represents heat treatment step and c represents cooling step.

### (Polarization Treatment)

The organic piezoelectric material of the present invention is employed in the ultrasonic transducer after polarization treatment. As a polarization treatment method in the polarization treatment in the present invention, there can be applied a method according to a well-known direct current voltage application treatment, alternating current voltage application treatment, or corona discharge treatment.

For example, the corona discharge treatment in the corona discharge treatment method can be carried out employing an apparatus available on the market composed of a high voltage power source and an electrode.

It is preferred that discharge conditions are properly selected, since they vary due to kind of an apparatus used or treatment ambience.

When the high voltage power source is used, the voltage is preferably from −1 to −20 kV, the current is preferably from 1 to 80 mA, the distance between the electrodes is preferably from 1 to 10 cm, and voltage applied is preferably from 0.5 to 2.0 MV/m.

The electrode used is preferably a needle electrode, a linear electrode (wire electrode) or a network electrode, each being conventionally used, but the present invention is not limited thereto.

### (Substrate)

The substrate used is selected according to usage of the organic piezoelectric material layer in the present invention. As the substrate in the present invention, there can be used a plate or film of a plastic such as polyimide, polyamide, polyimideamide, polyethylene terephthalate (PET), polyethylene naphthalate (PEN), polymethyl methacrylate (PMMA), a polycarbonate resin, or a cycloolefin polymer.

The substrate may be those in which the surface of these materials is covered with aluminum, gold, copper, magnesium, or silicon. The substrate may be a plate or a film of simple substance of aluminum, gold, copper, magnesium, silicon or a single crystal of a halide of rare earth element.

### (Electrode)

The ultrasonic transducer comprising an organic piezoelectric material in the present invention is one which is manufactured by forming an electrode on one or both sides of an organic piezoelectric material film (layer), and subjecting the piezoelectric material layer to polarization treatment.

The electrode is formed from electrode materials comprised mainly of gold (Au), platinum (Pt), silver (Ag), palladium (Pd), copper (Cu), nickel (Ni), or tin (Sn).

In the formation of the electrode, a layer of a metal such as titanium (Ti) or chromium (Cr) is formed according to a sputtering method as an under layer to obtain a thickness of from 0.02 to 1.0 μm.

Then, metal materials composed mainly of the metal elements described above or metal materials composed of alloys thereof, and optionally insulation materials are deposited on the under layer according to a sputtering method or another appropriate method to form a 1 to 10 μm thick layer.

The electrode formation can be carried out by a screen printing method employing a conductive paste in which fine metal particles are mixed with a low melting point glass, a dipping method or a melt splaying method

Further, a given voltage is applied across electrodes formed on both sides of a piezoelectric material layer to polarize the piezoelectric material layer. Thus, an ultrasonic transducer is obtained.

### (Ultrasonic Wave Probe)

The ultrasonic transducer of the present invention is employed in ultrasonic wave probe.

The ultrasonic wave probe of the present invention comprises an oscillator for ultrasonic wave transmission and an oscillator for ultrasonic wave reception, and the ultrasonic transducer of the present invention is preferably employed as an oscillator for reception.

In the present invention, one oscillator may bear both of ultrasonic wave transmission and reception, but it is preferred that an oscillator for ultrasonic wave transmission and an oscillator for ultrasonic wave reception are separately provided in a probe.

A piezoelectric material constituting an oscillator for ultrasonic wave transmission may be a conventional ceramics inorganic piezoelectric material or an organic piezoelectric material.

In the ultrasonic wave probe, the oscillator for ultrasonic wave reception can be disposed on an oscillator for transmission or in parallel.

Preferred embodiment is one having a structure that the oscillator for ultrasonic wave reception of the present invention is provided on an oscillator for ultrasonic wave transmission. In this case, the ultrasonic transducer of the present invention, which is laminated on another polymeric material (a film as a substrate of the polymer (resin) having a relatively low relative dielectric constant as described above, for example, a polyester film), may be provided on the oscillator for ultrasonic wave transmission.

It is preferred that the total thickness of the laminate of the oscillator and the polymeric material matches a preferable reception frequency band region in view of design of the probe.

The thickness is preferably from 40 to 150 μm in view of an ultrasonic wave medical diagnostic imaging system for practical use or actual frequency band used for collection of living body information.

The probe may be provided with a backing layer, an acoustic matching layer, an acoustic lens and the like. The probe may be one in which many oscillators having a piezoelectric material are two dimensionally arranged. The plural probes, being two dimensionally arranged, may constitute a scanner in which the plural probes conduct scanning in order, followed by imaging.

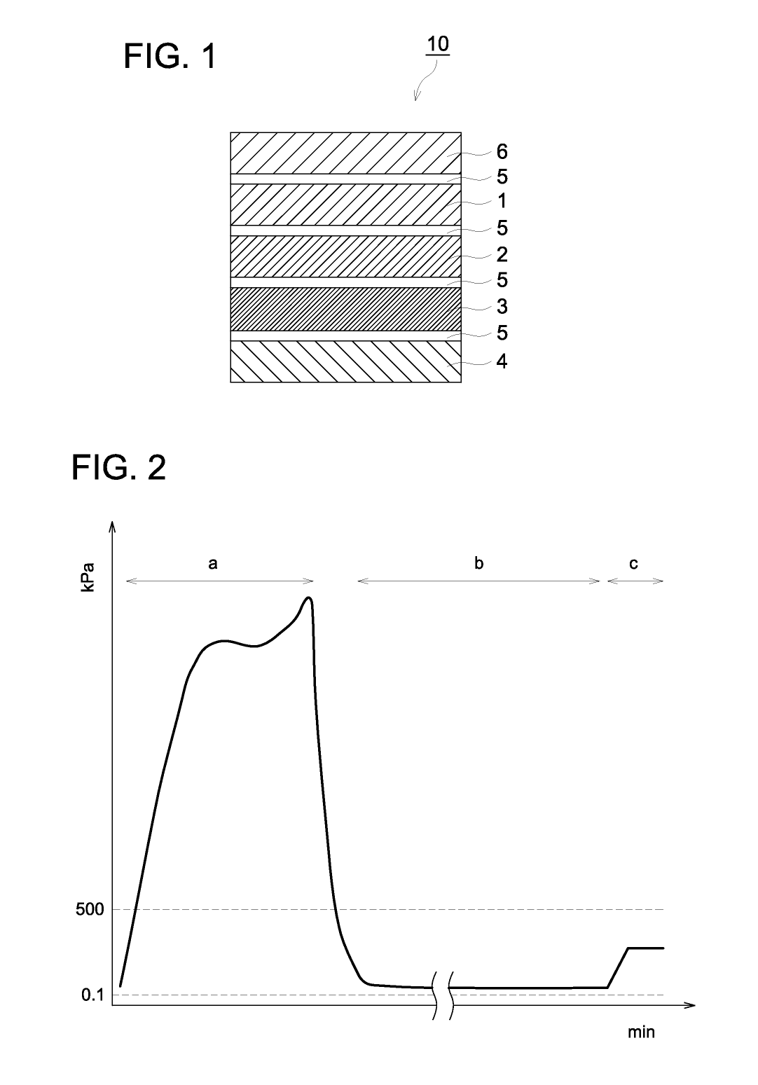

FIG. 1 is a schematic drawing showing the structure of the main section of an ultrasonic wave probe.

The ultrasonic wave probe 10 has two ultrasonic transducers which sandwiches substrate 2, where one ultrasonic transducer which comprises electrodes 5 on the both side of piezoelectric material for ultrasonic wave transmission 1 and the other ultrasonic transducer which comprises electrodes 5 on the both side of piezoelectric material for ultrasonic wave reception 3, and acoustic lens 6 and backing layer on the outside of each ultrasonic transducer, respectively.

### (Acoustic Lens)

The acoustic lens is arranged to focus the ultrasonic wave beam in terms of reflection, resulting in enhance resolution.

It is essentially required that the acoustic lens matches to acoustic impedance (density×sound velocity: (1.4−1.6)×10⁶ kg/m²·sec) of living body by adhering well to the living body and reduces reflection of ultrasonic wave, as well as focusing the ultrasonic wave, and also has small loss of ultrasonic wave as itself.

Namely, the acoustic lens is provided on the portion where it contacts to the body for focusing acoustic wave beam, which is made based on polymeric materials such as conventional rubber. The material for the lens is expected to have enough smaller sound velocity than that of human body, small acoustic attenuation and acoustic impedance close to that of skin in human living body.

When lens material has enough smaller sound velocity than that of human body, the lens may be formed convex shape, whereby it slips smoothly and in safety in case of making diagnosis. Small acoustic attenuation results in transmission and reception of ultrasonic wave in high sensitivity. Further, the acoustic impedance close to that of skin in human living body minimizes reflection, namely makes larger transmittance, resulting in transmission and reception of ultrasonic wave in high sensitivity as well.

As materials constituting the acoustic lens of the present invention, employable is conventional homopolymer such as silicone rubber, fluorine silicone rubber, or epichlorohydrin rubber; copolymer rubber such as ethylene-propylene copolymer rubber which is prepared by copolymerizing ethylene and propylene. Of these, silicone based rubber is preferably employed.

As silicone based rubber employable to the present invention, listed is silicone rubber or fluorine silicone rubber.

Of these, silicone rubber is preferable in view of characteristic for lens material. Silicone rubber is referred to as organopolysiloxane which has molecular skelton comprising Si—O bonds and a plurality of organic groups are mainly bonded to these Si atoms. Generally, main component thereof is methyl polysiloxane, where methyl group is 90% or more based on total organic groups. Ones in which hydrogen atom, phenyl group, vinyl group or aryl group is introduced instead of methyl group are also employable.

The silicone rubber can be obtained by kneading organo polysiloxane having high polymerization degree with hardner (vulcanizing agent) such as benzoyl peroxide, and by heating to vulcanize and harden.

Organic or inorganic filler such as silica and nylon powder and auxiliaries of vulcanization such as sulfur or zinc oxide may be added as appropriate.

Butadiene based rubbers include copolymerizing rubbers which have butadiene singly or is obtained by copolymerizing butadiene as main component with small amount of styrene or acrylonitril.

Of these, butadiene rubber is preferable in view of characteristic for lens material. Butadiene rubber is referred to as synthesized rubber by polymerizing butadiene having conjugated double bond.

Butadiene rubber can be prepared by 1,4- or 1,2-polymerizing butadiene singly having conjugated double bond. Butadiene rubber vulcanized by sulfur or the like may be employable.

One is also employable for the acoustic lens, which is obtained by blending silicone based rubber and butadiene based rubber and by vulcanizing to hardened.

For example, above rubber can be obtained by kneading silicone and butadiene in appropriate ratio by using kneading roll, followed by adding vulcanizing agent such as benzoyl peroxide, and by heating to vulcanize and harden.

In this case, it is preferable to add zinc oxide as auxiliaries of vulcanization. Zinc oxide promotes vulcanization without deteriorating lens characteristics, resulting in shortening vulcanization time.

Other than above, other additives may be added to the extent that the characteristics of colorant or acoustic lens are not impaired. In order to obtain one in which acoustic impedance is approximate to that of human body and sound velocity is smaller than human body and low attenuation, mixing ratio of silicone based rubber and butadiene based rubber is generally preferable 1:1, however this mixing ratio may be changeable as appropriate.

According to the purpose such as adjusting sound velocity or density, inorganic filler such as silica, alumina, titan oxide or organic resin such as nylon can be incorporated in the base (main component) comprising rubber material such as silicone based rubber described above.

### (Backing Layer)

In an ultrasonic wave probe, for the purpose of inhibiting propagation of ultrasonic wave to the rear side, it is preferable to provide backing layer which is arranged on the back side of the ultrasonic transducer. As the result, pulse width can be shortened.

### (Acoustic Matching Layer)

The acoustic matching layer (also referred to as “λ/4 layer”) is multiply arranged so as to minimize the difference of acoustic impedance between oscillator and living body, resulting in enhancing efficiency for transmission and reception of ultrasonic wave.

### (Ultrasonic Wave Medical Diagnostic Imaging System)

The above ultrasonic wave probe of the present invention can be applied to various ultrasonic wave diagnostic systems. For example, it can be suitably applied to an ultrasonic wave medical diagnostic imaging system equipped with an ultrasonic wave probe (probe) in which a piezoelectric material oscillator is arranged which transmits an ultrasonic wave to an examinee such as a patient and receives an ultrasonic wave reflected from the examinee as an echo signal.

Further, the ultrasonic wave medical diagnostic imaging system is preferably equipped with a transmission and reception circuit, which supplies an electric signal to the ultrasonic wave probe to generate ultrasonic wave and receives an echo signal which each piezoelectric material oscillator in the ultrasonic wave probe receives, and a transmission and reception control circuit, which controls transmission and reception of the transmission and reception circuit.

The system is further equipped with an image data conversion circuit which converts an echo signal which the transmission and reception circuit receives to an ultrasonic wave image data of an examinee. The system is equipped with a display control circuit, which controls a monitor with an ultrasonic wave image data converted by the image data conversion circuit and displays an image, and a control circuit, which controls the entire ultrasonic wave medical diagnostic imaging system.

The transmission and reception control circuit, the image data conversion circuit and the display control circuit are connected to the control circuit and the operation thereof is controlled through the control circuit.

An electric signal is applied to each piezoelectric oscillator in the ultrasonic wave probe to transmit an ultrasonic wave to an examinee and a reflection wave generated by acoustic impedance mismatch inside the examinee is received by the ultrasonic wave probe.

The transmission and reception circuit described above corresponds to “an electric signal generation means”, and the image data conversion circuit corresponds to “an image processing means”.

The ultrasonic wave diagnostic system as described above, comprising the oscillator for ultrasonic wave reception of the present invention which is excellent in piezoelectric characteristic and thermal resistance and is suitable for high frequency and broad band, can provide an ultrasonic wave image with improved image quality and reproduction stability as compared with a conventional one.

### EXAMPLES

Next, the present invention will be explained employing examples, but the present invention is not limited thereto.

### (Manufacture and Evaluation of Organic Piezoelectric Material)

### Example 1

Powder of a vinylidene fluoride copolymer having a vinylidene fluoride (hereinafter also referred to as VDF) and trifluoroethylene (hereinafter also referred to as 3FE) in molar ratio of 75:25 was dissolved in a mixture solvent of methyl ethyl ketone (hereinafter also referred to as MEK) and dimethyl formamide (hereinafter also referred to as DMF) having ratio of 9:1 at 50° C., and resulting solution was cast on a glass plate.

Subsequently, it was dried by evaporating the solvent at 50° C. to obtain a film (organic piezoelectric material) with a thickness of about 140 μm and melting point of 155° C.

The resulting film was stretched by a factor of 3.8 at room temperature by using mono-axial stretching machine having load cell which can measure load on the chuck.

After finishing primary stretching, chuck was shifted to the position whereby stress at the end of the film became to 20 kPa. Then, the film was heat treated at 135° C. for 1 hour while maintaining the stress at 100 kPa by controlling distance between chucks without releasing the film from chucks. Subsequently, the film was cooled down to the room temperature while distance between chucks was gradually relaxation treated to the stretching direction (secondary stretching treatment). The amount of secondary stretching (relaxation) was determined as 9% stretching based on the length of film after heat treatment.

The thickness of the resulting film after heat treatment was 43 μm. On both side of the resulting film, gold/aluminum was vapor deposited to have surface resistance of 20Ω or less to obtain the sample with the surface electrodes.

Then, the resulting sample was subjected to polarization treatment, while an alternating voltage of 0.1 Hz is applied to the electrodes. The polarization treatment was carried out while gradually increasing voltage from a low voltage to a final electric field between the two electrodes being 100 MV/m.

The final polarization amount was determined from the residual polarization by considering piezoelectric material as condenser, namely from amount of charge accumulation against thickness, area of electrode, and applied electric field, to obtain Sample 1 of the present invention.

In Table 1, listed were primary stretching factor, tension immediately after primary stretching, temperature of heat treatment, time of heat treatment, tension during heat treatment, and amount of secondary stretching (relaxation) during cooling step.

As other samples of the present invention and comparative samples, Samples 1-12 were prepared by forming film, forming electrode and polarizing as the same mariner as Sample 1.

### [Planarity of Organic Piezoelectric Material]

The resulting organic piezoelectric material with electrode was cut down in rectangles having 100 mm length for stretching direction and 20 mm in a direction perpendicular to stretching direction. The resulting cut piezoelectric film is placed on the transparent acryl plate, pressed by load of 10 kg/cm² (980 kPa) through metal plate. Planarity was evaluated by visually from acryl plate side.

A: No crease was noted and no cracks were noted in electrode and piezoelectric film.

B: No crease was noted but crack was noted in electrode and piezoelectric film and was practically problematic.

C: Crease was noted and crack was noted in electrode and piezoelectric film and was practically problematic.

### [Evaluation Method of Organic Piezoelectric Material Film]

The electrodes on both sides of each of the above-obtained samples with electrodes being connected by a lead, each sample was scanned at 25° C. with 600 frequencies of the same interval in the frequency range of from 40 Hz to 110 MHz, employing an impedance analyzer 4294A manufactured by Agilent Technologies, Inc. The relative dielectric constant at the thickness resonance frequency was determined.

Similarly, a peak frequency P of resistance and a peak frequency S of conductance approximately at the thickness resonance frequency were determined, and electromechanical coupling coefficient kₜ was determined according to the following equation. As the electromechanical coupling coefficient, 0.3 or more is practically preferable range.

kₜ=(α/tan(α))^{1/2} wherein α=(π/2)×(S/P)

A method of determining the electromechanical coupling coefficient from the thickness resonance frequency employing an impedance analyzer is in accordance with item 4.2.6 Thickness longitudinal Oscillation of Disk-shaped Oscillator described in electrical test method of piezoelectric ceramic oscillator in Japan Electronics and Information Technology Industries Association Standard JETTA EM-4501 (formerly, EMAS-6100). The evaluation results are shown in Table 1.

TABLE 1

| | Primary Stretching | | | Heat treatment | | | | | | | |
|---|---|---|---|---|---|---|---|---|---|---|---|
| | | Tension | | | | Tension | Secondary | | Electro- | | |
| | | after | Tension | | | during | Stretching | | mechanical | Planarity | |
| Sample | Factor | stretching*¹ | Release after | Temp. | Time | treatment*¹ | (relaxation) | Polarization | coupling | Visual | |
| No. | Times | kPa | Stretching | ° C. | hr. | kPa | % | mC/m² | coefficient | Rank | Remarks |
| 1 | 3.8 | 20 | None | 135 | 1.0 | 100 | +9 | 83 | 0.33 | A | Inv. |
| 2 | 2.6 | 20 | None | 135 | 1.0 | 20 | +5 | 85 | 0.34 | A | Inv. |
| 3 | 3.8 | 20 | None | 135 | 1.0 | 100 | — | 80 | 0.32 | C | Comp*³. |
| 4 | 3.8 | 20 | Done*² | 135 | 1.0 | 50 | +9 | 80 | 0.32 | C | Comp. |
| 5 | 3.8 | 20 | None | 135 | 1.0 | 0.01 | +9 | 80 | 0.32 | B | Comp. |
| 6 | 3.8 | 20 | None | 135 | 1.0 | 0.1 | +9 | 83 | 0.33 | A | Inv. |
| 7 | 3.8 | 20 | None | 135 | 1.0 | 50 | +9 | 80 | 0.32 | A | Inv. |
| 8 | 3.8 | 20 | None | 135 | 1.0 | 500 | +9 | 83 | 0.33 | A | Inv. |
| 9 | 3.8 | 20 | None | 135 | 1.0 | 750 | (Broken) | — | — | — | Comp. |
| 10 | 3.8 | 20 | None | 100 | 1.0 | 100 | +9 | 63 | 0.25 | A | Inv. |
| 11 | 2.6 | 20 | None | 140 | 0.5 | 50 | +9 | 85 | 0.34 | A | Inv. |
| 12 | 2.6 | 20 | None | 110 | 10.0 | 10 | +5 | 83 | 0.33 | A | Inv. |

*¹Tension is calculated by dividing detected load at stretching machine by measured area.
*²After stretching, tension was released once.
*³Cooling without secondary stretching.
Inv.: Inventive Example, Comp. Comparative Example

As is apparent from Table 1, samples prepared in the range of the present invention exhibit excellent planarity and piezoelectric characteristic. Further, it is found to prepare oscillator easily and to have excellent processing suitability.

### Example 2

### (Preparation and Evaluation of Ultrasonic Wave Probe)

CaCO₃, La₂O₃, Bi₂O₃ and TiO₂ were provided as component materials, and MnO as a subcomponent material. The component materials were weighed so that a final component composition was (Ca_{0.97}La_{0.03})Bi_{4.01}Ti₄O₁₅.

Subsequently, the materials were added with pure water, mixed for 8 hours in a ball mill charged with media made of zirconia, and then sufficiently dried to obtain a mixture powder.

The resulting mixture powder was temporarily molded and subjected to temporary calcination in air at 800° C. for 2 hours to obtain a preliminary calcination product.

Subsequently, the preliminary calcination product was added with pure water, pulverized in a ball mill charged with media made of zirconia, and then dried to obtain a piezoelectric ceramics material powder.

The pulverization time and the pulverization conditions during the pulverization being changed, a piezoelectric ceramics material powder having a particle size of 100 nm was obtained.

The piezoelectric ceramics material powder having a different particle size was added with 6% by mass of pure water as a binder, and press molded to obtain a preliminary plate-like molding having a thickness of 100 μm. The resulting preliminary plate-like molding was vacuum packed and then press molded by a pressure of 235 MPa.

The resulting preliminary plate-like molding was subjected to calcination to obtain a calcination product having a thickness of 20 μm as a final calcination product. The calcination temperature was 1100° C. An electric field of not less than 1.5×Ec (MV/m) being applied for 1 minute, the calcination product was subjected to polarization treatment.

### (Preparation of Laminate Oscillator for Reception)

The vinylidene fluoride copolymer film (organic piezoelectric material film) subjected to electron beam irradiation obtained in Example 1 was adhered to a 50 μm thick polyester film through an epoxy adhesive to obtain a laminate oscillator.

The resulting laminate oscillator was further subjected to polarization treatment in the same manner as above.

Subsequently, the resulting laminate oscillator for reception was laminated on the piezoelectric material for transmission described above according to an ordinary method, and further provided with a backing layer and an acoustic consistency layer. Thus, an ultrasonic wave probe was prepared.

An ultrasonic wave probe for comparison was prepared in the same manner as the ultrasonic wave probe obtained above, except that a laminate oscillator employing only the vinylidene fluoride copolymer film (organic piezoelectric material film) was used instead of the laminate oscillator for reception.

Subsequently, the two ultrasonic wave probes obtained above were evaluated for reception sensitivity and dielectric breakdown strength.

With regard to the reception sensitivity, a fundamental frequency f₁ of 5 MHz was transmitted, and then, relative reception sensitivity of a reception secondary harmonic f₂ of 10 MHz, a reception tertiary harmonic f₃ of 15 MHz and a reception quaternary harmonic f₄ of 20 MHz was determined.

The relative reception sensitivity was measured, employing a sound intensity measuring system Model 805 (1 to 50 MHz), manufactured by Sonora Medical System, Inc., 2021 Miller Drive Longmont, Colo. (0501 USA).

After the above probes were subjected to load test in which a load power increased to five times was applied for 10 hours, relative reception sensitivity of the resulting probes was measured and evaluated as a measure of the dielectric breakdown strength.

Sensitivity, lowering by not more than 1% of that before subjected to the load test, was evaluated as good. Sensitivity, lowering by less than 10% to more than 1% of that before subjected to the load test, was evaluated as accepted. Sensitivity, lowering by not less than 10% of that before subjected to the load test, was evaluated as unacceptable.

In the above evaluation, it proved that the probe with the reception piezoelectric (material) laminate oscillator of the present invention had relative reception sensitivity about 1.2 times that of the probe for comparison, and had high dielectric breakdown strength.

That is, it was confirmed that the oscillator for ultrasonic wave reception of the present invention was suitably applied to a probe used in the ultrasonic wave medical diagnostic imaging system.

### EXPLANATION OF TILE SYMBOLS

1. Piezoelectric material for reception
2. Substrate
3. Piezoelectric material for transmission
4. Backing layer
5. Electrode
6. Acoustic lens
10. Ultrasonic wave probe

1. A method for stretch-treating an organic piezoelectric material comprising sequential steps of:
primary stretching an organic piezoelectric material which has not been stretched;
heat treating the organic piezoelectric material subjected to primary stretching; and
secondary stretching the heat treated organic piezoelectric material while cooling down the organic piezoelectric materal to room temperature,
wherein:
tension is applied continuously to the organic piezoelectric material from the primary stretching step to the cooling step without releasing the tension, and
heat treatment is carried out while keeping the tension in a range of 0.1-500 kPa.

2. The method for stretching an organic piezoelectric material of claim 1,
wherein a heat treating temperature is in a range of 100 to 140° C., and
wherein a heat treating time is in a range of 30 minutes to 10 hours.

3. The method for stretching an organic piezoelectric material of claim 2, wherein primary stretching is carried out mono-axially or bi-axially by a stretching factor from 2 to 10, and secondary stretching is carried out by not more than 10% in a longitudinal direction.

4. The method for stretching an organic piezoelectric material of claim 1, wherein the organic piezoelectric material comprises a copolymer of vinylidene fluoride and triflioroethylene, and a content ratio of vinylidene fluoride and triflioroethylene is in the range of from 95 to 60 mol % and in the range of from 5 to 40 mol %, respectively.

5. A method for manufacturing the organic piezoelectric material comprising a step of:
polarizing the organic piezoelectric material manufactured by the method of claim 1.

6. An ultrasonic transducer comprising:
the organic piezoelectric material manufactured by the method of claim 5; and
an electrode.

7. An ultrasonic wave probe comprising the ultrasonic transducer of claim 6.

8. An ultrasonic wave medical diagnostic imaging system comprising:
an electric signal generating device;
an ultrasonic wave probe in which a plurality of oscillators are arranged which receive the electric signal and transmit an ultrasonic wave to an examinee and generate a reception signal according to a reflection wave returned from the examinee; and
an image processing device which forms an image of the examinee according to the reception signal generated by the ultrasonic wave probe,
wherein the ultrasonic wave probe comprises an oscillator for ultrasonic wave reception comprising a piezoelectric material for ultrasonic wave reception, and
wherein at least one of the ultrasonic transducers is the ultrasonic transducer of claim 6.
